(19) United States
(12) Patent Application Publication
(10) Pub. No.: US 2025/0312119 A1
(43) Pub. Date: Oct. 9, 2025

(54) SURFACE-FORMED FIDUCIAL MARKERS AND SURGICAL OBJECT TRACKING

(71) Applicants: Arthrex, Inc., Naples, FL (US); The Johns Hopkins University, Baltimore, MD (US)

(72) Inventors: Mathias Unberath, Baltimore, MD (US); Wenhao Gu, Baltimore, MD (US); Justin Opfermann, Baltimore, MD (US); Axel Krieger, Alexandria, VA (US); Jonathan Knopf, Naples, FL (US)

(73) Assignees: Arthrex, Inc., Naples, FL (US); The Johns Hopkins University, Baltimore, MD (US)

(21) Appl. No.: 18/630,226

(22) Filed: Apr. 9, 2024

(51) Int. Cl. A61B 90/00; A61B 34/00; A61B 34/20

(52) U.S. Cl. CPC A61B 90/39; A61B 34/25; A61B 2034/2055; A61B 2034/2065; A61B 2090/367; A61B 2090/373; A61B 2090/3916; A61B 2090/3933; A61B 2090/3941; A61B 2090/3979; A61B 2090/3995

(57) ABSTRACT

A method for tracking one or more features of an object in a surgical field includes applying a liquid compound for a cement to an object in the surgical field thereby affixing a cementitious fiducial marker to the object. Image data is captured at a first wavelength range in a visible spectrum and second image data at a second wavelength range in an infrared or near infrared spectrum. Features in the image data are identified in the first image data. Additionally, the fiducial markers are identified in the second image data. A relationship among the features of the at least one object and the fiducial markers are calculated. Based on the relationship, the positions of the features are identified based on the positions of the fiducial markers.

### BACKGROUND

The disclosure generally relates to a method for tracking one or more objects in a medical environment and, more particularly, relates to an image-based tracking system that utilizes flexibly applied fiducial markers to assist in object tracking. The tracking of objects in various settings may introduce a variety of challenges. While rigidly applied fiducial markers may improve tracking, such devices also introduce limitations in fixation and application flexibility. The disclosure provides for improved tracking methods that may be implemented in a variety of medical environments to improve the tracking of objects with various cameras or image sensors.

### SUMMARY

The disclosure provides for systems and methods that may assist in the tracking of one or more objects in a surgical or medical environment. In various implementations, the improved tracking may be supported by one or more fiducial markers, which may be positioned on a surface of an object in a surgical field. In various implementations, the fiducial markers may be applied to a rigid surface of the object via a liquid compound configured to adhere to and solidify in place in connection with the object throughout the surgical procedure. In operation, one or more image sensors of the disclosed system may capture image data in the surgical field to track the position and/or orientation of one or more features of the object. However, rather than detecting the features directly in the image data, the disclosed method may provide for the detection of the positions of the features of the object relative to the positions of the fiducial markers identified in the image data. In this way, the disclosure may provide for improved tracking of the object in the surgical field.

In various implementations, the fiducial markers may include a reflective or luminescent material that may illuminate or luminesce in response to receiving light within a first range of wavelengths. For example, a liquid compound used to form the fiducial markers may incorporate a luminescent material that becomes excited and emits a luminescent emission in response to receiving radiation in the near infrared (NIR) spectral range. The luminescent emissions output from the fiducial markers may similarly be output in the NIR spectral range, such that they may be readily identifiable via a compatible imager without creating distracting reflections in the visible light range of wavelengths. Accordingly, the disclosed method may provide for the tracking of the location and/or orientation of various features of the object responsive to changes in the position of the fiducial markers as detected by their luminescent positions in the NIR range. In this way, the positions of the one or more features of the objects apparent in the visible wavelengths of light may be identified and tracked based on the position and orientation of the fiducial markers applied to the object.

These and other features, objects and advantages of the present disclosure will become apparent upon reading the following description thereof together with reference to the accompanying drawings.

### BRIEF DESCRIPTION OF THE DRAWINGS

FIG. 1 is an environmental view of a surgical imaging system;

FIG. 2 is a process diagram demonstrating an object tracking method based on a plurality of fiducial markers formed on the surface of the object;

FIG. 3 is a process diagram demonstrating a method for tracking features in a surgical field with a camera over a visible light spectrum by monitoring fiducial markers in a near infrared spectrum;

FIG. 4 is a process diagram demonstrating a method for object tracking in a surgical field;

FIG. 5 is a projected view of a surgical site demonstrating an applicator applying a liquid compound to the surface of an object to form a plurality of fiducial markers;

FIG. 6 is a projected view of a surgical site demonstrating the application of a plurality of fiducial markers in an arthroscopic procedure;

FIG. 7 is an exemplary diagram demonstrating the tracking of an object in the form of a surgical tool with a plurality of fiducial markers; and

FIG. 8 is a block diagram demonstrating a surgical imaging system in accordance with the disclosure.

### DETAILED DESCRIPTION

In the following description, reference is made to the accompanying drawings, which show specific implementations that may be practiced. Wherever possible, the same reference numbers will be used throughout the drawings to refer to the same or like parts. It is to be understood that other implementations may be utilized and structural and functional changes may be made without departing from the scope of this disclosure.

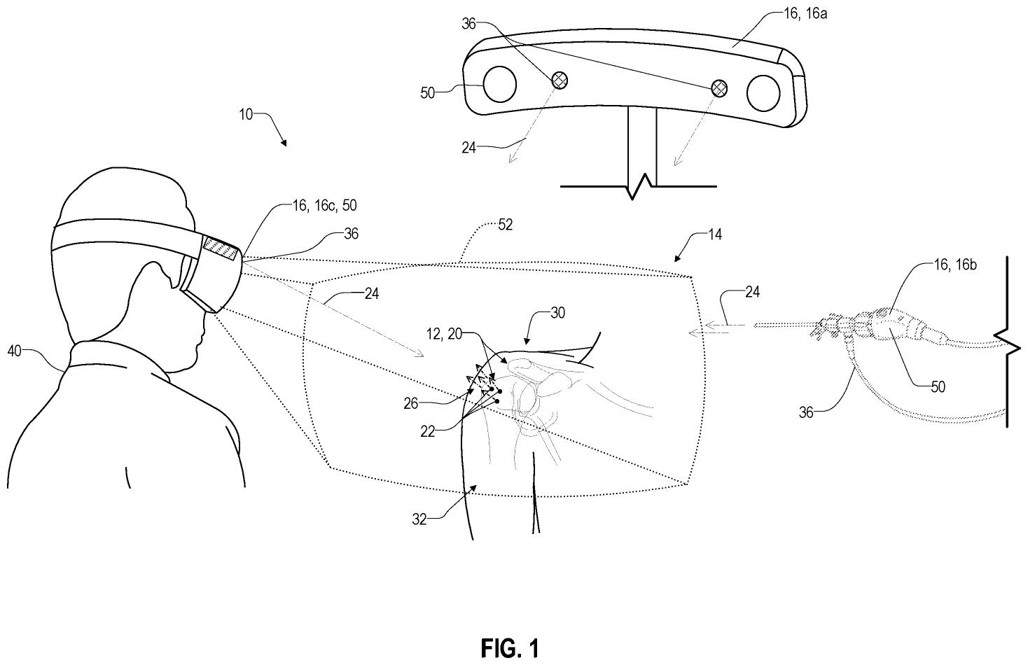

Referring to FIG. 1, the disclosure generally provides for a surgical imaging system 10 and corresponding methods for tracking one or more objects 12 in a surgical field 14. As shown, the surgical imaging system 10 may be implemented with a variety of imagers or cameras 16, which may include conventional monoscopic cameras, stereoscopic cameras 16a, arthroscopic or endoscopic cameras 16b, and/or cameras incorporated in one or more user display devices, for example, an integrated camera 16c incorporated in a head-mounted display 18. As discussed in the following detailed description, the camera 16 implemented with the system 10 may provide for the tracking of one or more visible features 20 of the objects 12 based on the detection and tracking of a plurality of fiducial markers 22 that may be applied to the objects 12 intraoperatively. The fiducial markers 22 may include a luminescent material that may become excited responsive to an excitation emission 24 comprising one or more wavelengths outside the visible light range and emit tracking emissions 26 that may also be substantially outside the visible light spectrum. In this configuration, the tracking emissions 26 may be detected by compatible imagers of the cameras 16 to accurately track a position and/or orientation of the objects 12 in the surgical field 14.

In the example shown in FIG. 1, the object 12 may correspond to an anatomy 30 or bony anatomy of a patient 32. As shown, the fiducial markers 22 may be affixed to a portion of the anatomy 30 of the patient 32. At least one light source 36 of the surgical imaging system 10 may output the excitation emission 24 in the NIR spectral range. Responsive to receiving the excitation emission 24, each of the fiducial markers 22 may emit luminescent tracking emissions 26 that may be detected in image data captured by the one or more cameras 16. The tracking emissions 26 may correspond to resulting luminescent emissions output from the luminescent material in the fiducial markers 22, which may similarly be emitted in the NIR spectral range. For example, the luminescent material in the fiducial markers 22 may correspond to a fluorescent dye, for example, indocyanine green (ICG), brilliant blue green (BBG), infracyanine green (IfCG), bromophenol blue (BPB), and iFluor® 790. In this way, the tracking emissions 26 may be detected by the one or more cameras 16, thereby providing readily identifiable markers to track the position and/or orientation of the object 12, in this case, the anatomy 30 of the patient 32. Further, the spectral range of the excitation and tracking emissions 24, 26 may ensure that the associated light necessary to track and detect the locations of the fiducial markers 22 may not distract a user or operator 40 of the imaging system 10.

As discussed herein, the image data captured by the cameras 16 may comprise visible light image data, including wavelengths from approximately 380 nm to 700 nm, and near infrared image data, ranging from approximately 760 nm to 2400 nm. Accordingly, one or more imagers 50 or imaging arrays of the cameras 16 may be configured to detect light in the visible light spectrum and the NIR spectrum. In this configuration, the imagers 50 of the cameras 16 may be configured to capture the light in the NIR spectrum in a range of wavelengths corresponding to the luminescent material or fluorescent dye incorporated in the fiducial markers 22. By capturing the light in the visible range and the NIR range, the system 10 may identify the positions of various features 20 of the object relative to the positions of the fiducial markers 22. In various implementations, the light sources 36 may comprise one or more emitters that may illuminate the surgical field 14 or the field of view 52 of the imagers 50 with light in the range of wavelengths necessary to excite the luminescent material of the fiducial markers 22 as the excitation emission 24. Accordingly, the light source(s) 36 and camera(s) 16 of the imaging system 10 may provide for the capture of visible light data in coordination with the capture of the tracking emissions 26 associated with the fiducial markers 22 throughout operation.

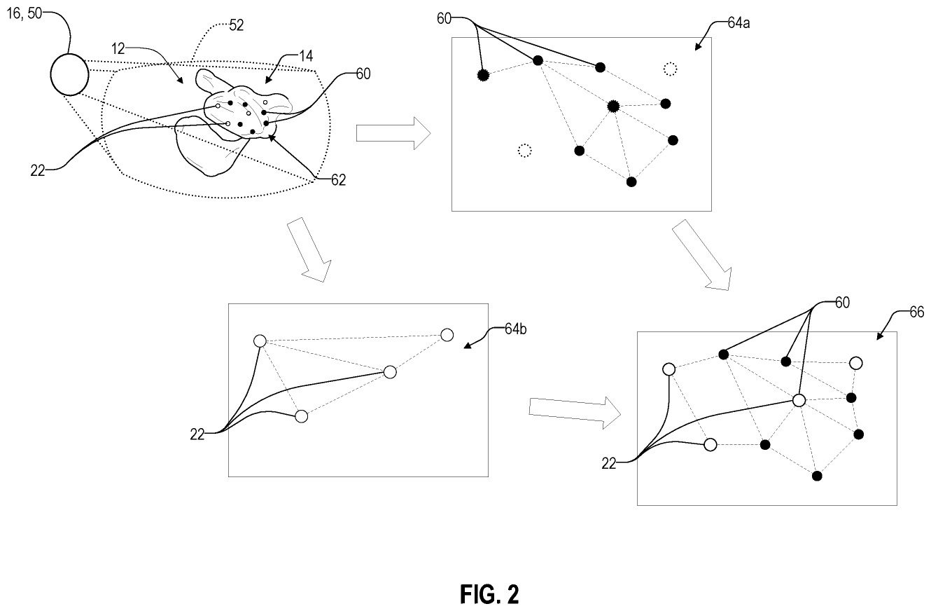

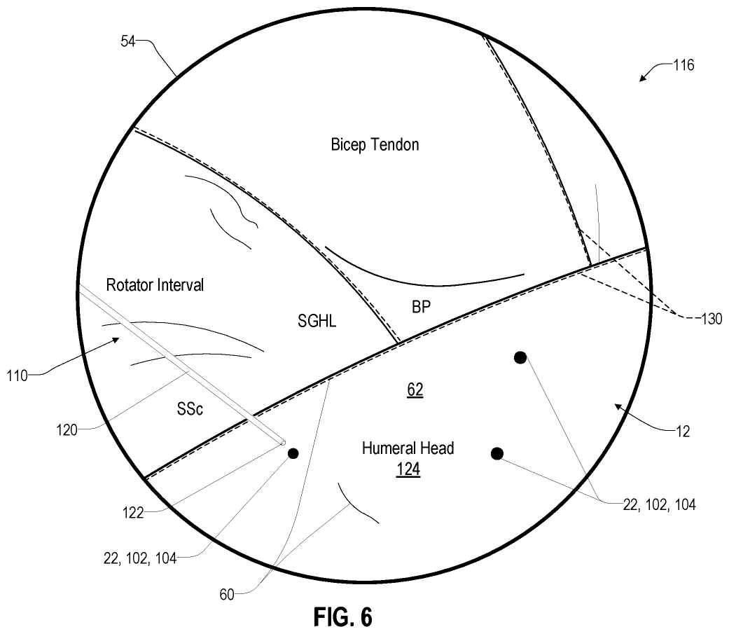

Referring now to FIG. 2, a tracking procedure for the fiducial markers 22 is discussed in further detail. As shown, image data representing the object 12 may be captured in a field of view 52 of the imager 50 of the camera 16. Throughout operation, the imagers 50 of the camera 16 may detect various features 60 of the object 12, which may be apparent in a visible spectrum of light over a surface 62 of the object 12 exposed to the field of view 52. Concurrently or in rapid succession, the fiducial markers 22 may be illuminated with the excitation emission 24 and the resulting luminescent emissions or tracking emissions 26 may be captured by the imagers 50 configured to detect light in the NIR light spectrum. Accordingly, the imagers 50 of the camera 16 of the system 10 may be configured to capture first image data 64a in the visible light spectrum depicting the features 60 of the object 12 as well as second image data 64b in the NIR spectrum depicting the locations of the fiducial markers 22 as identified by the tracking emissions 26. As shown in the example of FIG. 2, the positions of one or more of the fiducial markers 22 may coincide with the position of one or more of the features 60 on the surface of the object 62. In some cases, the positions of the fiducial markers 22 may be selected to coincide with one or more readily identifiable features of the object 12 (e.g., anatomical features, edges, functional elements, labels, etc.). In this way, the placement and formation of the fiducial markers may be provided to the operator/user based on known or identifiable features, as further discussed in reference to FIG. 6.

As further shown in FIG. 2, the first image data and second image data 64a, 64b may be combined to form composite image data 66, which may demonstrate the features 60 of the object 12 as well as the locations of the fiducial markers 22 in the same or overlapping and calibrated fields of view 52 of the camera 16. Once the locations of the features 60 of the object 12 are associated based on their relative positions and/or orientations to the fiducial markers 22, the features 60 of the objects 12 may be tracked in the surgical field 14 by detecting only the tracking emissions 26 associated with the fiducial markers 22. As the tracking emissions 26 may be more readily distinguishable over the surface 62 of the object 12, the tracking of the fiducial markers 22 may provide for improved tracking of the object 12.

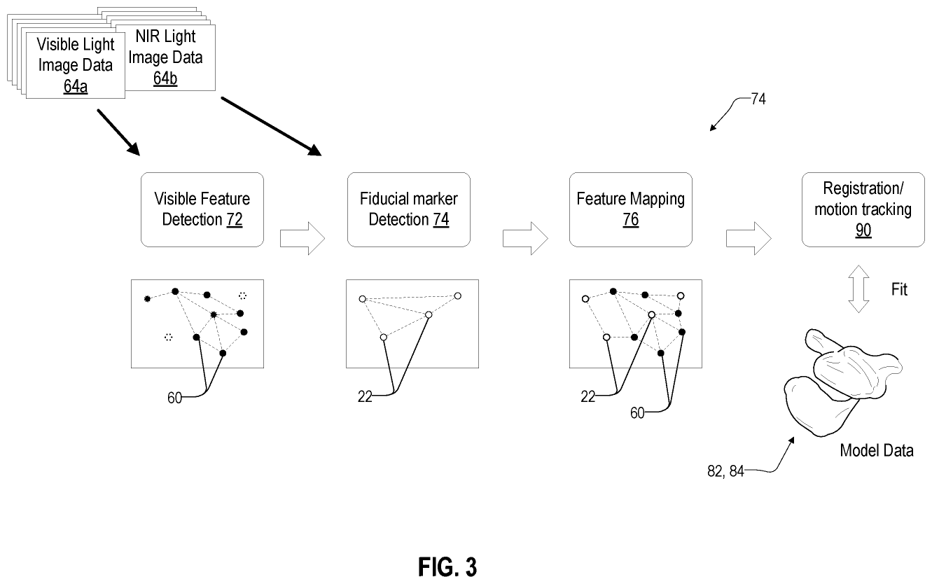

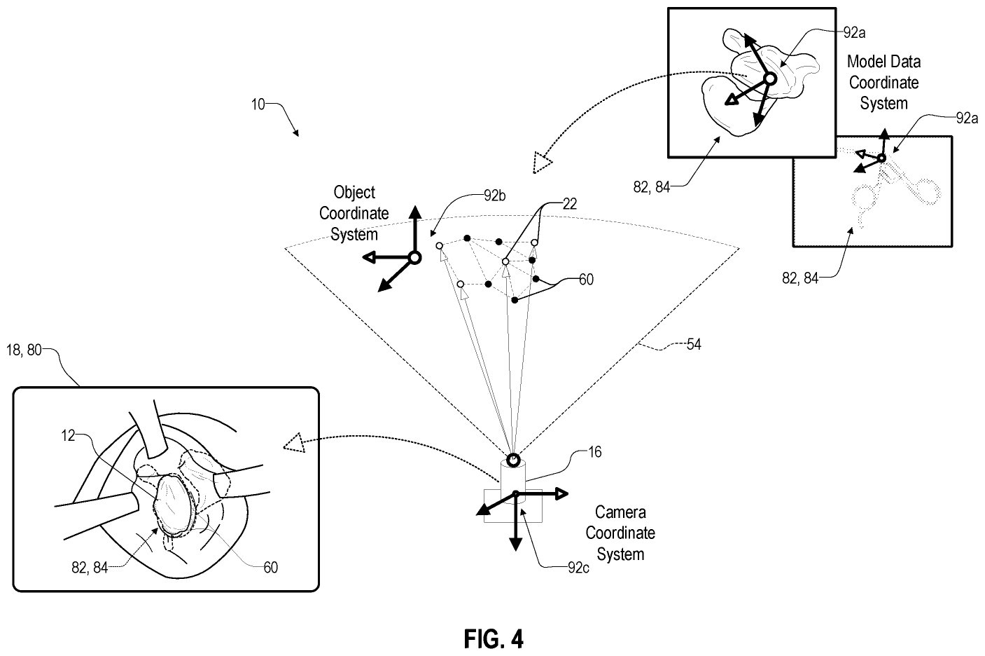

Referring to FIGS. 3 and 4, the method for tracking the object 12 in the image data 64 is described in further detail. As shown, the first image data 64a and second image data 64b may be captured by the one or more imagers 50 of the camera 16. The features 60 of the objects 12 may be detected by one or more controllers 70 (e.g., graphic processing units (GPU)) of the imaging system 10 by applying a feature recognition or detection routine 72. Similarly, the one or more controllers 70 may identify the positions of the fiducial markers 22 in the second image data 64b via a fiducial recognition routine 74. As discussed herein, the recognition routines may correspond to one or more image processing algorithms that may be configured to detect the features 60 and/or positions of the fiducial markers 22 via various image processing techniques (e.g., edge detection, threshold detection, boundary contrast or gradient detection, convolutional neural-networks, trained models, etc.).

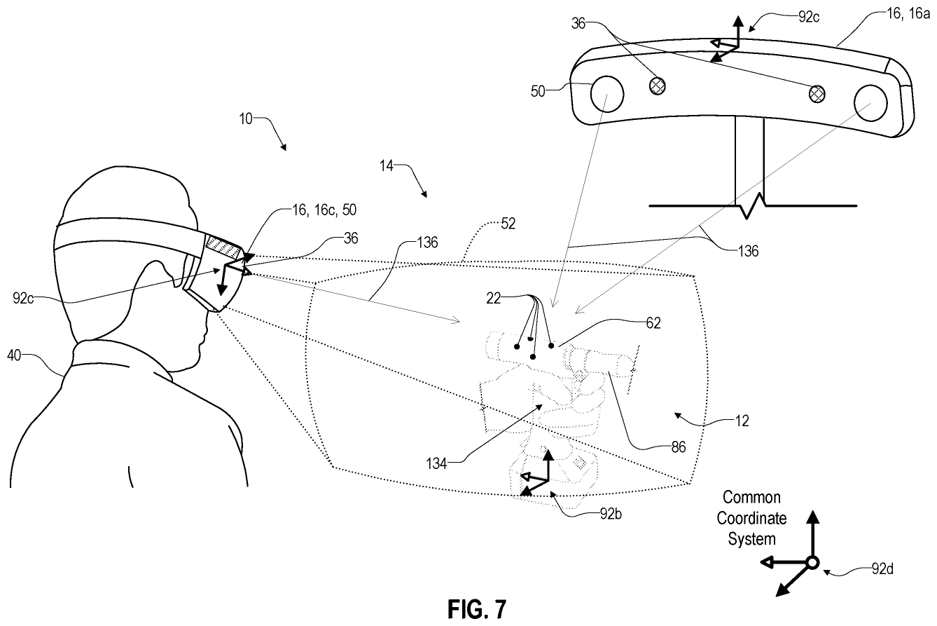

Once the features 60 and positions of the fiducial markers 22 are identified in the image data 64, a feature mapping routine 76 may identify the coordinates or positions of the features 60 relative to the fiducial markers 22. In this way, the position and/or orientation of the features 60 of the object 12 may be tracked by only viewing or monitoring the second image data 64b with the known relationship of the features 60 to the fiducial markers 22. Following the feature mapping routine 76, the imaging system 10 may track the position and orientation of the object 12 in the surgical field 14 based on the readily identifiable tracking emissions 26 output from the fiducial markers 22. In this way, the system may provide for tracking of the object 12 throughout the surgical field 14 as well as additional advanced features that may provide for improved visualization of the object for the user 40. As discussed herein, the detection of the tracking emissions 26 in the second image data 64b is described as being applied to detect the corresponding locations of the features 60 of the object 12. However, in some cases, the positions of the features 60 of the object 12 may also be tracked in the first image data 64a to assist tracking the positions of the fiducial markers 22. Such selective or hybrid tracking of the features 60 in the first image data 64a and the fiducial markers 22 in the second image data 64b may be particularly beneficial in cases where a line of sight of the object 12 becomes partially occluded from the field of view 52 as described in further detail in reference to FIG. 7.

Still referring to FIGS. 3 and 4, in some implementations, the imaging system 10 may further provide for advanced visualization techniques that may be implemented with a display 80 (e.g., a conventional display panel, transparent display, etc.) and/or the head-mounted display 18. In the example shown, enhanced visualization data 82 of the object 12 may be aligned with the features 60 of the object 12 to provide for enhanced augmented visualization of the surgical field 14. For example, the visualization data 82 may include model data 84 or scan data that may provide three-dimensional details or enhanced information related to the object 12, the patient 32, and/or a procedure. As shown, the model data 84 is a bone model of the patient 32. However, the model data 84 or visualization data 82 may relate to various forms of patient or procedural information, information related to a surgical tool 86, or related to various surgical accessories or objects that may be present in the surgical field 14.

In cases where the enhanced visualization data 82 is displayed, the controller 70 may register the visualization data 82 or model data 84 of the object 12 to the corresponding features 60 identified in the first image data 64a via a registration routine 90. The registration routine 90 may include one or more steps identifying features 60 of the object 12 in the first image data 64a and aligning the features 60 with corresponding portions of the visualization data 82. The registration routine 90 may be implemented as an iterative fit process, wherein the corresponding coordinates or reference points of the visualization data 82 are repeatedly oriented via a fitting or matching routine until a best fit solution is identified aligning the visualization data 82 with the corresponding features 60 of the object 12. In this way, a model coordinate system 92a of the visualization or model data 82, 84 may be aligned with an object coordinate system 92b of the object 12.

As further demonstrated in FIG. 4, the object coordinate system 92b may be tracked based on the second image data 64b and the corresponding locations of the fiducial markers 22 identified by the tracking emissions 26. In this way, the tracking emissions 26 may be detected by the controller 70 from the second image data 64b, such that the object coordinate system 92b may be tracked. With the visualization data 82 or model data 84 registered to the object 12 (e.g., the model coordinate system 92a registered to the object coordinate system 92b), the relative positions of the features 60 of the object 12 may be aligned within a camera coordinate system 92c responsive to the relative positions of the fiducial markers 22 in the field of view 52. In this way, the enhanced visualization data 82 or model data 84 may be displayed superimposed over the corresponding anatomy 30 on one or more of the displays 18, 80. In this way, the imaging system 10 may provide for the execution of one or more enhancement routines to display the visualization data 82 or model data 84 of the object 12 superimposed over or positioned relative to the corresponding features 60 of the object 12 on one of the displays 18, 80 for improved surgical navigation.

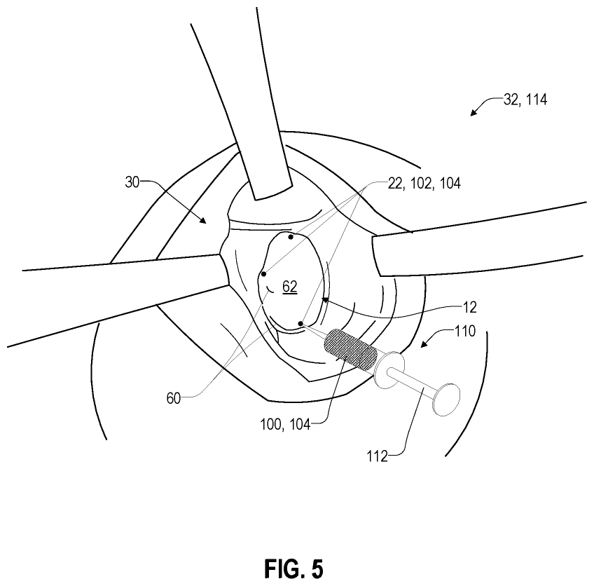

Referring now to FIG. 5, a method for forming the plurality of fiducial markers is discussed in further detail. As previously discussed, the fiducial markers 22 may be formed by a liquid compound 100 which may be in the form of a bone cement 102, for example, a calcium phosphate cement (CPC), a polymethyl methacrylate (PMMA) cement, or similar materials. In some implementations, the cement 102 may correspond to a biocompatible or bioresorbable material comprising the luminescent material 104 intermixed throughout. Depending on the application, the viscosity of the cement 102 may vary. For example, for an endoscopic procedure as illustrated in FIG. 6, the viscosity may be high enough to avoid mixing with a distension fluid (e.g., saline) prior to fixation and hardening to the surface 62 of the object 12. Once applied and cured, the locations of the resulting fiducial markers 22 may remain fixed to the surface 62 of the object 12 throughout a surgical procedure or observation period.

In liquid form 100, the cement 102 or cement compound may be applied to the surface 62 of the object 12 with an applicator 110, which may be in the form of a syringe. Accordingly, the fiducial markers 22 may be formed by depressing a plunger 112, thereby causing the liquid compound 100 to form on the surface 62 of the object 12. In the example shown, the surface 62 may correspond to a surface of a bone (e.g., a glenoid cavity) that may be exposed during a procedure 114. In contrast, as later demonstrated in FIG. 6, the fiducial markers 22 may be formed on the surface 62 of an enclosed object 12 during a closed procedure 116. In each case, the liquid compound 100 may be applied to the surface 62 and allowed to cure or harden and adhere to the surface 62. Depending on the application, the proportions of the fiducial markers 22 may vary but may generally correspond to circular or oval shapes formed on the surface 62. In general, the dimensions of the fiducial markers 22 may range from approximately 0.25 mm to approximately 5 mm, more particularly, from approximately 1 mm to approximately 3 mm in proportion. Once cured to the surface 62 of the object 12, the fiducial markers 22 may serve as fixed reference points in relation to the features 60 of the objects 12 that may be readily identifiable based on the tracking emissions 26 output in the NIR light spectrum.

Referring now to FIG. 6, the formation of the fiducial markers 22 is discussed in reference to the closed procedure 116. In the example shown, the fiducial markers 22 are formed on the surface 62 of a rigid or bony portion of the anatomy 30 of the patient 32. In the example shown, the liquid compound 100 is delivered through a distal tip 120 of a syringe needle 122. Once affixed to the surface 62 of the object 12, the fiducial markers 22 may serve as reference points that may orient the image data 64 captured within the field of view 52 relative to the corresponding anatomy 30 of the patient 32.

In the example shown, the anatomy 30 is exemplified as a humeral head 124 of a humorous of the patient 32. Similar to the examples previously discussed, the humeral head 124 and/or various features 60 of the object 12 may be identified via the feature recognition routine 72. Following the identification of the features 60, the positions of the fiducial markers 22 may be identified by the fiducial recognition routine 74 and the locations/orientations of the features 60 may further be mapped relative to the positions of the fiducial markers 22 via the feature mapping routine 76. Once mapped in relation to the features 60, the identification of the fiducial markers 22 in the second image data 64b may define the relative positions of the features 60, which may provide for alignment of the enhanced visualization data 82 and/or model data 84. In the example shown in FIG. 6, the portions of the first image data 64a that are visible within the field of view 52 are annotated with the visualization data 82 identifying the anatomy 30 of the patient 32. Additionally, the visualization data 82 is included in the form of dividing lines 130 between the included portions of the anatomy 30. The dividing lines 130 or segmentation lines are demonstrated superimposed over the boundaries of the corresponding tissue. Throughout navigation of the camera 16, in this case the endoscopic camera 16b, the annotations or enhanced visualization data 82 and/or model data 84 may be updated and repositioned in response to the locations of the fiducial markers 22 as determined from the tracking emissions 26. In this way, the imaging system 10 may provide for improved navigation and identification of various positions of the anatomy 30 of the patient 32.

Referring now to FIG. 7, yet another exemplary application of the fiducial markers 22 is shown in reference to a surgical tool 86. In the example shown, the surgical tool 86 may correspond to a drill or pin driver along which an axial alignment for a surgical procedure may be defined. In operation, the fiducial markers 22 may similarly be formed with the applicator 110, such that the tracking emission 26 may be readily identified within the field of view 52 of the camera 16. In this case, the exemplary camera 16 is a stereoscopic camera 16a. In operation, the camera 16 may track various features 60 of the object 12 based on their positions in the surgical field 14 relative to the fiducial markers 22. In the example of the stereoscopic camera 16a or other camera technologies that may provide for depth detection or depth identification of the surfaces of the features 60 depicted in the field of view 52, the relative proportions of the object 12 may be identified based on the image data 64 without supplemental model data 84. In this case the surgical tool 86, the camera 16a may identify a three-dimensional profile similar to the model data 84 in addition to detecting the position or orientation of the object 12. In this way, the three-dimensional contours of the object 12, as well as the position and orientation of the object coordinate system 92b, may be tracked based on the detected positions of the fiducial markers 22 and tracking emissions 26 in the second image data 64b. Accordingly, once the fiducial markers 22 are mapped to the corresponding features 60 or model data 84 of the object 12 via the feature mapping routine 76, the orientation and position of the object 12 may be tracked within the field of view 52 based on only the detected positions of the tracking emissions 26 identifying the fiducial markers 22.

As shown in FIG. 7, the fiducial markers 22 are distributed over the surface 62 of the surgical tool 86 in a position away from an engagement surface 134 of the surgical tool 86. Additionally, the fiducial markers 22 are distributed over the surface 62 of the tool 86 within a line of sight 136 of one or more of the cameras 16. In the example shown, the head-mounted display 18 may include the integrated camera 16c in addition to the stereoscopic camera 16a. In such implementations, the position and orientation of the fiducial markers 22 and the corresponding features 60 of the surgical tool 86 may be tracked within a common coordinate system 92d within the surgical field 14. In this way, the surgical imaging system 10 may leverage the capture of the image data 64 from multiple perspectives via a plurality of cameras 16 to ensure that the fiducial markers 22 are tracked throughout the operation of the surgical tool 86 or, more generally, various objects 12 as discussed herein.

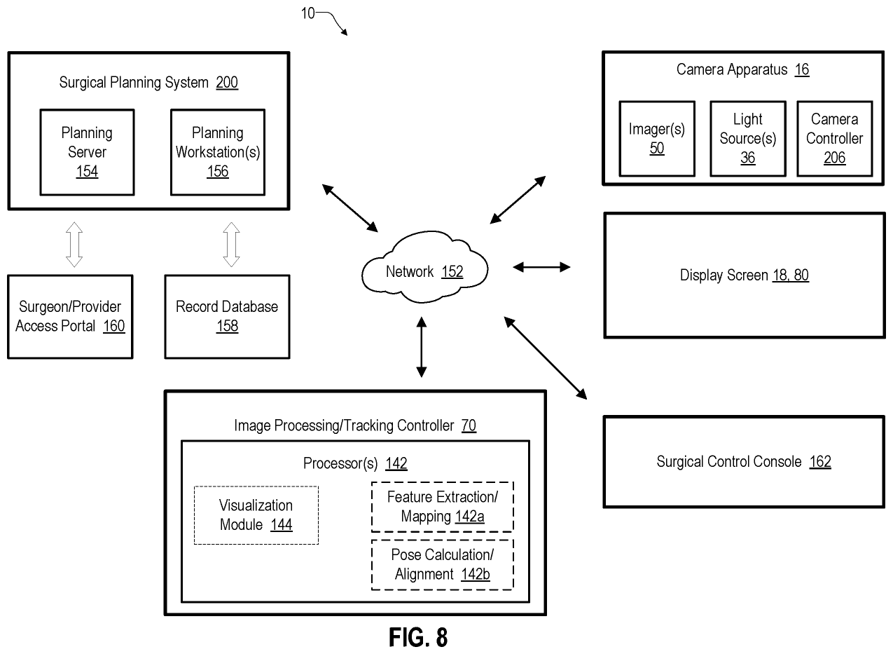

Referring now to FIG. 8, a block diagram of the visualization system 10 is shown. As previously discussed, the system 10 may include one or more cameras 16, which may include conventional monoscopic cameras, stereoscopic cameras 16a, arthroscopic or endoscopic cameras 16b, and/or cameras incorporated in one or more user display devices, for example, an integrated camera 16c incorporated in a head-mounted display 18. The one or more cameras 16 may include a plurality of imagers 50 or composite imaging devices that may capture the image data 64 including the visible light first image data 64a, including wavelengths from approximately 380 nm to 700 nm, and near infrared second image data 64b, ranging from approximately 760 nm to 2400 nm. Accordingly, the one or more imagers 50 or imaging arrays of the cameras 16 may be configured to detect light in the visible light spectrum and the NIR spectrum. By capturing the light in the visible range and the NIR range, the system 10 may identify the positions of various features 20 of the object relative to the positions of the fiducial markers 22.

In the example shown, the system 10 includes the controllers 70 denoted as an image processing and tracking controller 70. As previously discussed, the controller 70 may incorporate one or more processors 142, including one or more graphic processors (GPUs) that may be implemented for a feature extraction module 142a or one or more computational processing units (CPUs) that may provide for pose-calculation and alignment module 142b. The pose-calculation and alignment module 142b may generally be implemented to calculate and track the position of the features 60 of the object 12 relative to the fiducial markers 22. Additionally, in some cases the pose-calculation and alignment module 142b may be implemented to calculate a position and orientation or pose of the camera 16 relative to the anatomy 30 and/or align or calculate offsets among the various coordinate systems 54 as discussed herein.

In addition to processing the image data 64 (e.g., the visible light image data 64a and the NIR image data 64b), the controller 70 may be implemented to generate, access, and/or manipulate the visualization data 82 or model data 84, which may be in the form of various images and/or graphics generated or modified by a visualization module 144. In operation, each of the processors 212 and corresponding modules 142a, 142b, 144 may access local memory devices (not shown) and/or remote memory and/or databases associated with a surgical planning system 150 to access patient data, procedural steps, instructions, surgical guides, patient models (e.g. three-dimensional models from scans), or various surgical or medical information that may be associated with a patient and/or surgical procedure. Accordingly, the image processing controller 70 may be implemented in various configurations to support the operation of the visualization system 10. In various examples, the image data 64 and the visualization data 82 or model data 84 may be displayed on one or more displays 18, 80 as discussed herein. Each of the corresponding devices (e.g., cameras 16, displays 18, 80, controllers 70, etc.) may be in communication via a device network 152.

In general, various devices and components of the visualization system 10 and the surgical planning system 150 may incorporate a wide variety of specialty or general-purpose computational units and corresponding memory devices that may be communicatively accessed to process the various routines and access the corresponding information and/or data required to operate the visualization system 10. For example, the one or more processing units of the system 10 may include one or more graphic processing units, associated processing units, programmable arrays, and/or various computational circuits that may be programmed to facilitate the operations discussed herein. Similarly, the various memory devices accessed by the processing units may correspond to various forms of computer-readable storage media, such as random access memory (RAM), flash memory, read-only memory (ROM), programmable ROM (PROM), or similar forms of non-transitory, machine-readable storage media. Accordingly, the various operations of the controllers, processors, and/or servers as discussed herein may be implemented or enabled by utilizing a wide variety of processing units and corresponding memory devices, each of which may be selected based on the particular application associated with the underlying operation.

As previously discussed, the visualization system 10 may also be in communication with the surgical planning system 150, which may incorporate a planning server 154. The planning system 150 may incorporate various planning workstations 156 utilized to generate various surgical plans and process the patient data that may be used to generate the visualization data 82 and/or model data 84. The patient data as discussed herein may be stored in a patient record database 158, which may be populated with a variety of patient information including medical history, scanning information, procedural plans, etc. In various implementations, the patient data may correspond to one or more bone models and corresponding information that may be obtained via various medical scanning devices, such as computerized tomography (CT), magnetic resonant imaging (MRI) machines, and/or X-ray machines. Based on the patient data, an operator or computerized routine of each of the planning workstations 156 may generate surgical plans for operations associated with the patient data based on the specific type of procedure, implant, anatomic morphologies, or specific techniques associated with the surgical procedure for implementation with the visualization system 10. Once prepared, a surgical plan may be generated by the surgical planning system 150 and stored in the planning server 154 for access by the visualization system 10. Additionally, the surgical planning system 150 may provide for a surgeon or provider access portal 160, which may provide controlled access to one or more surgical plans for a specific patient or a group of patients associated with a surgeon or provider. Via the access portal 160, the surgeon or provider may view, revise, and/or make various updates to the surgical plan preoperatively in preparation for a specific procedure or group of procedures. In this way, the surgical planning system 150 may provide for assisted surgical planning while also supporting customization by the surgeon or medical professional 40 for implementation of the visualization system 10.

Finally, in various implementations, the device network 152 may further be in communication with one or more surgical control consoles 162. The surgical control consoles 162 may correspond to control devices or controllers for various surgical devices including, but not limited to, electric cautery tools, ablation probes, resection tools (e.g., shavers, drills, saws, etc.), surgical pumps (e.g., in-flow/out-flow pumps, etc.), insufflation devices, and/or various imaging or visualization devices (e.g., endoscopes, arthroscopes, laparoscopes, etc.). Accordingly, the visualization system 10 may be flexibly configured to support various steps of surgical procedures, including the operation of various surgical tools or devices that may be in communication with the device network 152 via the one or more surgical consoles 162.

The implementations described in the disclosure may be implemented in hardware, software, firmware, middleware, microcode, or any combination thereof. For a hardware implementation, the processing unit may include one or more ASICs (Application Specific Integrated Circuits), DSPs (Digital Signal Processing), DSPDs (DSP Devices), PLDs (Programmable Logic Devices), FPGAs (Field-Pro Grammable Gate Array), general-purpose processor, controller, microcontroller, microprocessor, other electronic unit, or combination thereof for performing the functions described in this disclosure.

For a software implementation, the techniques described in the embodiments of this disclosure can be implemented through modules (e.g., processes, functions, etc.) that perform the functions described in the embodiments of this disclosure. The software codes are stored in memory and executed by the processor. Memory can be implemented within the processor or external to the processor.

The device network as discussed herein could be any local area network (LAN), wireless local area network (WLAN), Intranet, Extranet, or any other appropriate architecture or system that facilitates communications in a network environment. The device network may further include any suitable communication link to such as wireless technologies (e.g., IEEE 802.11, 802.16, Wi-Fi, etc.), cellular technologies (e.g., 3G, 4G, etc.), etc., or any combination thereof. The device network may also include configurations capable of transmission control protocol/Internet protocol (TCP/IP) communications, user datagram protocol/IP (UDP/IP), or any other suitable protocol, where appropriate and based on particular needs.

According to some aspects of the disclosure, a method for tracking one or more features of an object in a surgical field, the method comprising: affixing a plurality of fiducial markers in fixed fiducial positions on the object; capturing first image data at a first wavelength range in a visible spectrum and second image data at a second wavelength range in an infrared or near infrared spectrum in a field of view; identifying the at least one feature of the object associated with a medical procedure in the first image data in the field of view; identifying the plurality of fiducial markers in the second image data in the field of view; calculating a spatial relationship between the at least one feature and the plurality of fiducial markers; and determining at least one of a feature position and a feature orientation of the object in response to fiducial positions of the fiducial markers based on the spatial relationship.

According to various aspects, the disclosure may implement one or more of the following features or configurations in various combinations:
the plurality of fiducial markers is formed by applying a liquid compound to the object, wherein the liquid compound cures forming the plurality of fiducial markers rigidly affixed to the object.
the fiducial marker comprises a luminescent material that reflects a nonvisible wavelength in the second wavelength range;
the luminescent material absorbs and fluoresces light in the NIR spectrum;
the luminescent material is a fluorescent dye comprising at least one of indocyanine green, brilliant blue green, infracyanine green, bromophenol blue, and iFluor® 790;
illuminating the field of view with light in the second wavelength range;
accessing surgical data comprising at least one of an image of the object, a model of the object, a scan of the object, or a surgical plan associated with the object;
displaying the surgical data in alignment with the feature position and feature orientation of the object based in response to the fiducial position and fiducial orientation of the at least one fiducial marker based on the spatial relationship;
the surgical data is shown aligned with the at least one object and superimposed over a scene corresponding to the field of view on a display screen of a head-mounted display;
the surgical data is three-dimensional scan data of the object;
the object comprises at least one of a surgical tool or instrument;
the object is a patient and the at least one feature comprises an anatomic feature visible in the first image data;
the at least one fiducial marker is affixed to a bony portion of the patient presented in the second image data;
the bony portion comprises an exposed bone surface accessed via an open procedure; and/or
the bony portion comprises an enclosed bone surface positioned within a patient cavity accessed via a closed surgical procedure.

According to another aspect of the disclosure, surgical imaging system comprising: at least one camera configured to capture first image data at a first wavelength range in a visible spectrum and second image data at a second wavelength range in an infrared or near infrared spectrum in a field of view; and at least one controller configured to receive the first image data and the second image data, the at least one controller configured to: identify at least one feature of an object associated with a medical procedure in the first image data; identify a plurality of fiducial markers affixed to the object in the second image data, wherein the fiducial markers are present in the field of view with the at least one feature of the object; calculate a spatial relationship between the at least one object and the plurality of fiducial markers; and determine at least one of a feature position and a feature orientation of the at least one feature in response to fiducial positions of the fiducial markers based on the spatial relationship.

According to various aspects, the disclosure may implement one or more of the following features or configurations in various combinations:
a cementitious fiducial marker is applied as a liquid compound and comprises a luminescent material that reflects a nonvisible wavelength in the second wavelength range;
at least one light source configured to emit light in the second wavelength range;
the controller is further configured to access surgical data comprising at least one of an image of the object, a model of the object, a scan of the object, or a surgical plan associated with the object;
the surgical data is three-dimensional scan data of the object;
the controller is further configured to register the surgical data in alignment with the object in response to the fiducial positions based on the spatial relationship;
the object comprises at least one of a surgical tool or instrument;
the object is a patient and the object comprises an anatomic feature visible in the first image data;
at least one fiducial marker is affixed to a bony portion of the patient presented in the second image data;
a fiducial applicator configured to deliver the liquid compound to a surface of the bony portion of the patient;
the fiducial applicator is a syringe or tube that delivers the liquid compound responsive to an applied pressure; and/or
the at least one camera is an endoscopic camera configured to capture the first image data and the second image data in a patient cavity.

According to yet another aspect of the disclosure, a method for tracking one or more features of an object in a surgical field, the method comprising: applying a liquid compound comprising a luminescent material that reflects an infrared or near infrared spectrum, wherein the liquid compound cures on the object in the surgical field forming a plurality of fiducial markers to the object in fiducial positions; capturing first image data in a visible spectrum in a field of view of a camera; capturing second image data in the infrared or near infrared spectrum in the field of view; identifying the at least one feature of the object in the first image data; identifying the plurality of fiducial markers in the second image data; calculating a spatial relationship between the at least one feature and the plurality of fiducial markers; and tracking at least one of a feature position and a feature orientation of the object in response to the fiducial positions of the fiducial markers based on the spatial relationship.

It will be understood that any described processes or steps within described processes may be combined with other disclosed processes or steps to form structures within the scope of the present device. The exemplary structures and processes disclosed herein are for illustrative purposes and are not to be construed as limiting.

It is also to be understood that variations and modifications can be made on the aforementioned structures and methods without departing from the concepts of the present device, and further it is to be understood that such concepts are intended to be covered by the following claims unless these claims by their language expressly state otherwise.

The above description is considered that of the illustrated embodiments only.

Modifications of the device will occur to those skilled in the art and to those who make or use the device. Therefore, it is understood that the embodiments shown in the drawings and described above are merely for illustrative purposes and not intended to limit the scope of the device, which is defined by the following claims as interpreted according to the principles of patent law, including the Doctrine of Equivalents

1. A method for tracking one or more features of an object in a surgical field, the method comprising:
affixing a plurality of fiducial markers in fixed fiducial positions on the object;
capturing first image data at a first wavelength range in a visible spectrum and second image data at a second wavelength range in an infrared or near infrared spectrum in a field of view;
identifying the at least one feature of the object associated with a medical procedure in the first image data in the field of view;
identifying the plurality of fiducial markers in the second image data in the field of view;
calculating a spatial relationship between the at least one feature and the plurality of fiducial markers; and
determining at least one of a feature position and a feature orientation of the object in response to fiducial positions of the fiducial markers based on the spatial relationship.

2. The method according to claim 1, wherein the plurality of fiducial markers is formed by applying a liquid compound to the object, wherein the liquid compound cures forming the plurality of fiducial markers rigidly affixed to the object.

3. The method according to claim 1, wherein the fiducial markers comprise a luminescent material that reflects a nonvisible wavelength in the second wavelength range.

4. The method according to claim 3, wherein the luminescent material absorbs and fluoresces light in the near infrared spectrum.

5. The method according to claim 3, wherein the luminescent material is a fluorescent dye comprising at least one of indocyanine green, brilliant blue green, infracyanine green, bromophenol blue, and iFluor® 790.

6. The method according to claim 1, further comprising:
illuminating the field of view with light in the second wavelength range.

7. The method according to claim 1, further comprising:
accessing surgical data comprising at least one of an image of the object, a model of the object, a scan of the object, or a surgical plan associated with the object.

8. The method according to claim 7, further comprising:
displaying the surgical data in alignment with the feature position and feature orientation of the object based in response to the fiducial position and fiducial orientation of the at least one fiducial marker based on the spatial relationship.

9. The method according to claim 8, wherein the surgical data is shown aligned with the at least one object and superimposed over a scene corresponding to the field of view on a display screen of a head-mounted display.

10. The method according to claim 7, wherein the surgical data is a three-dimensional representation of the object.

11. The method according to claim 1, wherein the object comprises at least one of:
a surgical tool or instrument; and
a patient and the at least one feature comprises an anatomic feature visible in the first image data.

12. The method according to claim 11, wherein the at least one fiducial marker is affixed to a bony portion of the patient presented in the second image data.

13. The method according to claim 11, wherein the bony portion comprises an exposed bone surface accessed via an open procedure.

14. The method according to claim 11, wherein the bony portion comprises an enclosed bone surface positioned within a patient cavity accessed via a closed surgical procedure.

15. A surgical imaging system comprising:
at least one camera configured to capture first image data at a first wavelength range in a visible spectrum and second image data at a second wavelength range in an infrared or near infrared spectrum in a field of view; and
at least one controller configured to receive the first image data and the second image data, the at least one controller configured to:
identify at least one feature of an object associated with a medical procedure in the first image data;
identify a plurality of fiducial markers affixed to the object in the second image data, wherein the fiducial markers are present in the field of view with the at least one feature of the object;
calculate a spatial relationship between the at least one object and the plurality of fiducial markers; and
determine at least one of a feature position and a feature orientation of the at least one feature in response to fiducial positions of the fiducial markers based on the spatial relationship.

16. The system according to claim 15, wherein a fiducial marker is applied as a liquid compound forming a cement, the cement comprising a luminescent material that emits a nonvisible wavelength in the second wavelength range.

17. The imaging system according to claim 15, further comprising:
at least one light source configured to emit light in the second wavelength range.

18. The imaging system according to claim 15, wherein the controller is further configured to:
access surgical data comprising at least one of an image of the object, a model of the object, a scan of the object, or a surgical plan associated with the object.

19. The imaging system according to claim 18, wherein the surgical data is three-dimensional scan data of the object.

20. The imaging system according to claim 18, wherein the controller is further configured to:
register the surgical data in alignment with the object in response to the fiducial positions based on the spatial relationship.

21. A method for tracking one or more features of an object in a surgical field, the method comprising:
applying a liquid compound comprising a luminescent material that reflects an infrared or near infrared spectrum, wherein the liquid compound cures on the object in the surgical field forming a plurality of fiducial markers on the object in fiducial positions;
capturing first image data in a visible spectrum in a field of view of a camera;
capturing second image data in the infrared or near infrared spectrum in the field of view;
identifying the at least one feature of the object in the first image data;
identifying the plurality of fiducial markers in the second image data;
calculating a spatial relationship between the at least one feature and the plurality of fiducial markers; and
tracking at least one of a feature position and a feature orientation of the object in response to the fiducial positions of the fiducial markers based on the spatial relationship.
